(19) United States
(12) Patent Application Publication
(10) Pub. No.: US 2008/0221107 A1
(43) Pub. Date: Sep. 11, 2008

(54) Therapeutic Agents

(75) Inventors: Fabrizio Giordanetto, Molndal (SE); Tord Inghardt, Molndal (SE); Peter Nordberg, Molndal (SE)

Correspondence Address: Pepper Hamilton LLP, 400 Berwyn Park, 899 Cassatt Road, Berwyn, PA, 19312-1183, US

(73) Assignee: ASTRAZENECA AB, Sodertalje (SE)

(21) Appl. No.: 11/995,570

(22) PCT Filed: Jul. 13, 2006
(86) PCT No.: PCT/SE2006/000878

§ 371 Date: Jan. 14, 2008

(30) Foreign Application Priority Data
Jul. 15, 2005 (SE) 0501688-6
Aug. 24, 2005 (SE) 0501879-1
Nov. 23, 2005 (SE) 0502568-9

(51) Int. Cl. A61K 31/53; C07D 513/04; A61K 31/519; A61K 31/5025; A61P 3/10; A61P 3/04; A61P 25/00

(52) U.S. Cl. 514/241; 544/220; 514/260.1; 544/278; 544/235; 514/248

(57) ABSTRACT

Compounds of formula (I), processes for preparing such compounds, their use in the treatment of obesity, psychiatric disorders, cognitive disorders, memory disorders, schizophrenia, epilepsy, and related conditions, and neurological disorders such as dementia, multiple sclerosis, Parkinson's disease, Huntington's chorea and Alzheimer's disease and pain related disorders and to pharmaceutical compositions containing them.

### FIELD OF INVENTION

The present invention relates to certain compounds of formula I, to processes for preparing such compounds, to their use in the treatment of obesity, psychiatric and neurological disorders, and to pharmaceutical compositions containing them.

### BACKGROUND OF THE INVENTION

Melanin concentrating hormone (MCH) is a cyclic peptide that was first isolated from fish over 15 years ago. In mammals, MCH gene expression is localised to the ventral aspect of the zona inserta and the lateral hypothalamic area (Breton et al., Molecular and Cellular Neurosciences, vol. 4, 271-284 (1993)). The latter region of the brain is associated with the control of behaviours such as eating and drinking, with arousal and with motor activity (Baker, B., Trends Endocrinol. Metab. 5: 120-126(1994), vol. 5, No. 3, 120-126 (1994)). Although the biological activity in mammals has not been fully defined, recent work has indicated that MCH promotes eating and weight gain (U.S. Pat. No. 5,849,708). Thus, MCH and its agonists have been proposed as treatments for anorexia nervosa and weight loss due to AIDS, renal disease, or chemotherapy. Similarly, antagonists of MCH can be used as a treatment for obesity and other disorders characterised by compulsive eating and excessive body weight. MCH projections are found throughout the brain, including the spinal cord, an area important in processing nociception, indicates that agents acting through MCHr1, such as compounds of formula I, will be useful in treating pain.

Two receptors for MCH (MCH receptor 1 (MCHr1) (Shimomura et al. Biochem Biophys Res Commun Aug. 11,2002;261(3):622-6) & MCH receptor 2 (MCH2r) (Hilol et al. J Biol Chem. Jun. 8,2001;276(23):20125-9)) have been identified in humans, while only one (MCHr1) is present in rodent species (Tan et al. Genomics June 2002;79(6):785-92). In mice lacking MCHr1, there is no increased feeding response to MCH, and a lean phenotype is seen, suggesting that this receptor is responsible for mediating the feeding effect of MCH (Marsh et al. Proc. Natl. Acad. Sci. USA, Mar. 5,2002;99(5):3240-5). In addition, MCHr1 antagonists have been demonstrated to block the feeding effects of MCH (Takekawa et al. Eur. J Pharmacol. Mar. 8,2002;438(3):129-35), and to reduce body weight & adiposity in diet-induced obese rats (Borowsky et al. Nature Med. Aug. 2002;8(8):825-30). The conservation of distribution and sequence of MCHr1 suggest a similar role for this receptor in man and rodent species. Hence, MCH receptor antagonists have been proposed as a treatment for obesity and other disorders characterised by excessive eating and body weight.

WO 2005/042541 discloses 3-(4-aminophenyl)thienopyrimid-4-one derivatives as MCHr1 antagonists for the treatment of obesity, diabetes, depression and anxiety.

WO 2005/047293 discloses 3-pyrrolidin-3-yl)thienopyrimid-4-one derivatives as MCHr1 antagonists for the treatment of obesity, diabetes, depression and anxiety.

WO 2005/103039 discloses 3-amino-pyrrolidinyl-substituted 3-(pyridin-3-yl)-thieno-pyrimid-4-one and 6-(pyrid-3-yl)-thienopyridazin-7-one derivatives as MCHr1 antagonists for treatment of obesity, anxiety, depression and other diseases.

There is an unmet need for MCH receptor antagonists that are more potent, more selective, more bioavailable and produce less side effects than known compounds in this field.

### SUMMARY OF THE INVENTION

It is an object of the present invention to provide compounds, which are useful in treating obesity and related disorders, psychiatric disorders, neurological disorders and pain. This object has been reached in that a compound of formula I have been provided for use as a MCH receptor antagonist.

According to another aspect of the invention a pharmaceutical formulation is provided comprising a compound of formula I, and a pharmaceutically acceptable adjuvant, diluent or carrier.

According to a further aspect of the invention, the use of a compound of formula I is provided, in the preparation of a medicament for the treatment or prophylaxis of conditions associated with obesity.

According to yet another aspect of the invention, a method is provided of treating obesity, psychiatric disorders, anxiety, anxio-depressive disorders, depression, bipolar disorder, ADHD, cognitive disorders, memory disorders, schizophrenia, epilepsy, and related conditions, and neurological disorders and pain related disorders, comprising administering a pharmacologically effective amount of a compound of Formula I to a patient in need thereof.

According to another aspect of the invention, a process for the preparation of compounds of formula I is provided.

According to a further aspect of the invention, a method is provided of treating obesity, type II diabetes, Metabolic syndrome and prevention of type II diabetes comprising administering a pharmacologically effective amount of a compound of formula I to a patient in need thereof.

### DESCRIPTION OF THE INVENTION

The invention relates to compounds of the general formula (I)

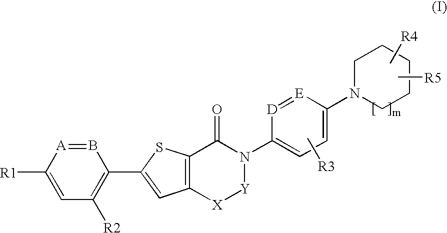

wherein A and B independently represent C or N.

D and E independently represent C or N,

X—Y represents N═C (provided that at least one of A, B, D or E represents N), or

X—Y represents C═N, or

X—Y represents N═N,

R¹ and R² independently represent H, C₁₋₃ alkyl (optionally substituted with one or more F), C₁₋₃ alkoxy (optionally substituted with one or more F), Cl or F,

R³ represents H, F, Cl, cyano, hydroxy, C₁₋₃ alkoxy (optionally substituted with hydroxy, methoxy or with one or more F) or C₁₋₃ alkyl (optionally substituted with hydroxy, methoxy, amino, methylamino, dimethylamino or with one or more F),

R⁴ and R⁵ independently represent H, oxo, hydroxy, Q-3 alkoxy (optionally substituted with hydroxy, methoxy or with one or more F), C₁₋₃ alkyl (optionally substituted with hydroxy, methoxy, amino, methylamino, dimethylamino or with one or more F) or C₁₋₃ acyloxy, the alkyl portion of which may optionally be substituted by one or more of methyl, amino, methylamino, dimethylamino or carboxy, m is 0 or 1

and tautomers, optical isomers and racemates thereof as well as pharmaceutically acceptable salts thereof.

The invention also relates to compounds of the general formula (Ia)

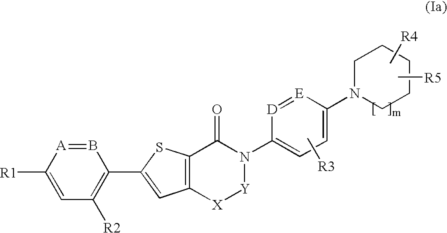

wherein A and B independently represent C or N,

D and E independently represent C or N,

X—Y represents N═C provided that at least one of A, B, D or E represents N), or

X—Y represents C═N, or

X represents NH and Y represents C═O, or

X—Y represents N═N,

R¹ and R² independently represent H, C₁₋₃ alkyl (optionally substituted with one or more F), C₁₋₃ alkoxy (optionally substituted with one or more F), Cl or F,

R⁴ represents H, F, Cl, hydroxy, C₁₋₃ alkoxy (optionally substituted with hydroxy, methoxy or with one or more F) or C₁₋₃ alkyl (optionally substituted with hydroxy, methoxy, amino, methylamino, dimethylamino or with one or more F),

R⁴ and R⁵ independently represent H, oxo, hydroxy, hydroxymethyl, C₁₋₃ alkoxy (optionally substituted with one or more F) or C₁₋₃ acyloxy,

m is 0 or 1,

and tautomers, optical isomers and racemates thereof as well as pharmaceutically acceptable salts thereof.

Particular groups now follow in which some of A, B, D, E, X, Y, m, R¹, l², R³, R⁴ and R⁵ in compounds of formula I-Ia are further defined. It will be understood that such group definitions may be used where appropriate with any of the other group definitions, claims or embodiments defined hereinbefore or hereinafter.

In one particular group of compounds of formula I-Ia,

X—Y represents C═N, or

X—Y represents N═N.

In another particular group of compounds of formula I-Ia,

A, B and E all represent C, and D represents N.

In another particular group of compounds of formula I-Ia,

X—Y represents N═C (provided that at least one of A, B, D or E represents N).

In another particular group of compounds of formula I-Ia,

A and B both represent C,

D and E both represent C,

X—Y represents C═N, or

X—Y represents N═N.

In yet another group of compounds of formula I-Ia,

A, B, D and E all represent C,

X—Y represents N═N,

R¹ represents Cl, F, CF₃, CHF₂, CH₂F, methyl, OCF₃ or OCHF₂,

R¹ represents H, Cl, F or CH₃,

R³ represents H, F, Cl, hydroxy, methoxy or hydroxymethyl,

where the R³ substituent is placed in the meta position relative to the fused heterocyclic ring system,

R⁴ represents oxo, hydroxy, methoxy or hydroxymethyl,

m is 0, and wherein the R⁴ substituent is placed in position 3 of the pyrrolidine ring and R⁵ represents H

In a further group of compounds of formula I-Ia,

A, B, and E represent C, and D represents N

X—Y represents N═C or C-N

R¹ represents Cl, F, CF₃, CHF₂, CH₂F, methyl, OCF₃ or OCHF₂,

R² represents H

R³ represents H

R⁴ represents hydroxy or hydroxymethyl,

m is 0, and wherein the R⁴ substituent is placed in position 3 of the pyrrolidine ring and R⁵ represents H or methyl placed in the same position as R⁴.

The term “pharmaceutically acceptable salt” refers to pharmaceutically acceptable acid addition salts. A suitable pharmaceutically acceptable salt of a compound of Formula I-Ia is, for example, an acid-addition salt of a compound of Formula I-Ia which is sufficiently basic, for example an acid-addition salt with an inorganic or organic acid such as:

(1S)-(+)-10-camphorsulfonic acid; cylohexylsulfamic acid; phosphoric acid; dimethylphosphoric acid; p-toluenesulfonic acid; L-lysine; L-lysine hydrochloride; saccharinic acid; methanesulfonic acid; hydrobromic acid; hydrochloric acid; sulphuric acid; 1,2-ethanedisulfonic acid; (±)-camphorsulfonic acid; ethanesulfonic acid; nitric acid; p-xylenesulfonic acid; 2-mesitylenesulfonic acid; 1,5-naphthalenedisulfonic acid; 1-naphthalenesulfonic acid; 2-naphthalenesulfonic acid; benzenesulfonic acid; maleic acid; D-glutamic acid; L-glutamic acid; D,L-glutamic acid; L-arginine; glycine; salicylic acid; tartaric acid; fumaric acid; citric acid; I,(−)-malic acid; D,L-malic acid and D-gluconic acid.

Throughout the specification and the appended claims, a given chemical formula or name shall encompass all tautomers, all stereo and optical isomers and racemates thereof as well as mixtures in different proportions of the separate enantiomers, where such isomers and enantiomers exist, as well as pharmaceutically acceptable salts thereof. Isomers may be separated using conventional techniques, e.g. chromatography or fractional crystallisation. The enantiomers may be isolated by separation of racemate for example by fractional crystallisation, resolution or HPLC. The diastereomers may be isolated by separation of isomer mixtures for instance by fractional crystallisation, HPLC or flash chromatography. Alternatively the stereoisomers may be made by chiral synthesis from chiral starting materials under conditions, which will not cause racemisation or epimerisation, or by derivatisation, with a chiral reagent. All stereoisomers are included within the scope of the invention.

Compounds of the present invention are intended to be chemically stable and it is assumed that it is within the skilled persons knowledge to identify which combinations of the above-defined groups in Formula I-Ia that may result in chemically unstable compounds of Formula I-Ia.

Some compounds of the Formula I-Ia, however, are intended to undergo metabolism in vivo to form an active species. Such compounds (prodrugs) contain a functional group (e.g. an ester) which may be hydrolysed to an alcohol (the active species) by the action of plasma and/or liver enzymes.

The following definitions shall apply throughout the specification and the appended claims.

Unless otherwise stated or indicated, the term “alkyl” denotes either a straight chain or branched alkyl group. Examples of said alkyl include methyl, ethyl, n-propyl, isopropyl, cyclopropyl, n-butyl, iso-butyl, sec-butyl and t-butyl. Preferred alkyl groups are methyl, ethyl, propyl, isopropyl and tertiary butyl.

Unless otherwise stated or indicated, the term “alkoxy” denotes a group O-alkyl, wherein alkyl is as defined above.

Unless otherwise stated or indicated, the term “acyloxy” denotes a group 0-acyl, wherein the term “acyl” denotes a group alkyl C(O).

Specific compound of the invention includes:
6-(4-chlorophenyl)-3-{4-[(3R)-3-hydroxypyrrolidin-1-yl]-3-methoxyphenyl}thieno[3,2-d][1,2,3]triazin-4(3B)-one;
6-(4-chlorophenyl)-3-{3-(hydroxymethyl)-4-[(3R)-3-hydroxypyrrolidin-1-yl]phenyl}thieno[3,2-d][1,2,3]triazin-4(3B)-one;
6-(4-chlorophenyl)-3-{6-[(3R)-3-hydroxypyrrolidin-1-yl]pyridin-3-yl}thieno[3,2-d]pyrimidin-4(3B)-one;
6-(4-chlorophenyl)-3-{5-[(3R)-3-hydroxypyrrolidin-1-yl]pyridin-2-yl}thieno[3,2-d]pyrimidin-4(3B)-one; and
6-(4-chlorophenyl)-3-{5-[3-hydroxy-3-methylpyrrolidin-1-yl]pyridin-2-yl}thieno[3,2-d]pyrimidin-4(3B)-one;
2-(4-chlorophenyl)-6-{5-[(3R)-3-hydroxypyrrolidin-1-yl]pyridin-2-yl}thieno[2,3-d]pyridazin-7(6H)-one,
and tautomers, optical isomers and racemates thereof as well as pharmaceutically acceptable salts thereof.

Methods of Preparation

The compounds of the invention may be prepared as outlined below according to any of the following methods. However, the invention is not limited to these methods, the compounds may also be prepared as described for structurally related compounds in the prior art.

Compounds of formula I-Ia, in which X—Y represents N═N, may be prepared by reacting, at room temperature, a compound of formula II

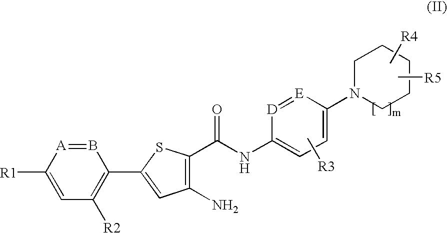

in which R¹, R², R³, R⁴, R⁵, A, B, D, E and m are as previously defined, with an diazotizing agent such as sodium or potassium nitrite or t-butyl nitrite in a solvent or solvent mixture containing acetic acid (75-100%) and water (0-25%), followed by alkaline aqueous work up, a method described e.g. in Daidone, G. et al. Heterocycles 43(11), 2385-96 (1996).

Compounds of formula Ia, in which X represents NH and Y represent C(O), may for example be prepared by reacting a compound of formula III with an aryl isocyanate IV, in analogy with methodology described in Graveleau, N. et al. Synthesis no 11, 1739-43 (2003).

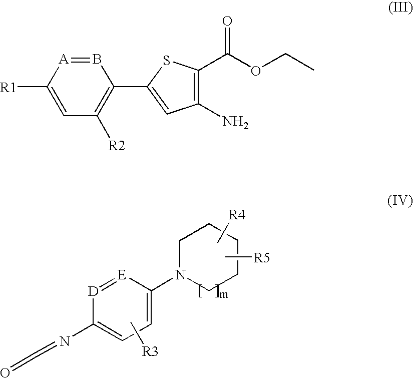

Compounds of formula I-Ia, in which X—Y represents C═N, may for example be prepared by condensing, in refluxing EtOH followed by heating (of the intermediate hydrazone) in HOAc, a compound of formula V with an aryl hydrazine VI, in analogy with methodologies described in Baraldi, P. G. et al. Nucleosides & Nucleotides 17(12), 2165-73 (1998) and in Marquet, J-P. et al. Tetrahedron 29, 435-39 (1973)

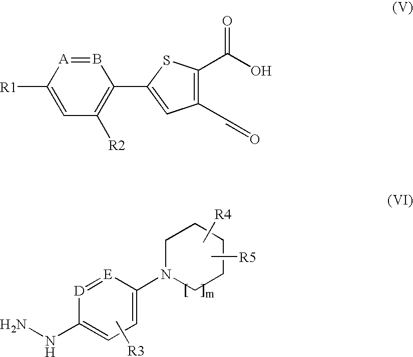

Alternatively, compounds of formula I-Ia, in which X—Y represents C═N, may be prepared by N-arylation of compounds of formula VII with haloaryl compounds of formula VIII, wherein Hal represents Cl, Br or I, at a temperature in the range of 0C to 250° C., preferably in the range of 50° C. to 160° C. in an inert solvent, for example toluene or dioxane in the presence of a catalytic cross-coupling system for example Cu₂O or CuI and trans-1,2-bis(methylamino)cyclohexane, and optionally in the presence of a base such as K₃PO₄ or CS₂CO₃.

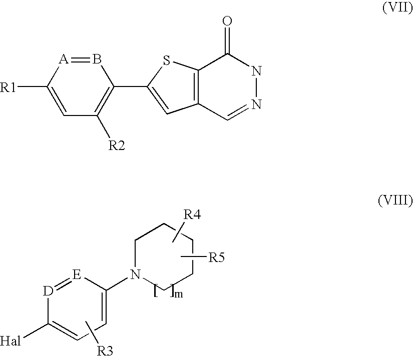

Compounds of formula IIa, in which X—Y represent N═C (and in which at east one of A,B,D or E represents N) may for example be prepared by reacting a compound of formula IX with a compound of formula X, in analogy with methodology described in WO2003/033476. Preferably, this reaction is carried out in a microwave reactor at 80-150° C. using EtOH, MeOH or phenol as solvent.

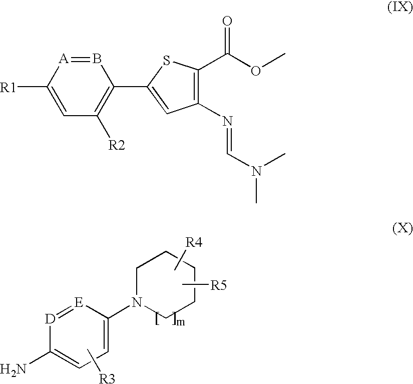

Alternatively, compounds of formula I-Ia may be prepared via a Suzuki or a Stille coupling reaction of a compound of formula XI with a compound of formula XII

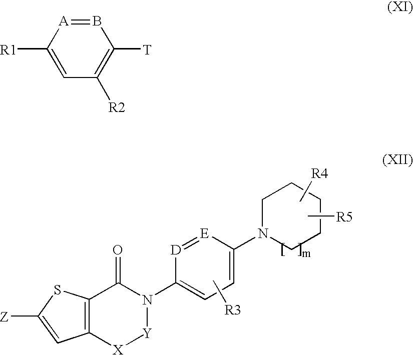

in which T represents B(OH)₂ or Sn(alkyl)₃ and Z represents a suitable leaving group such as I, Br or triflate.

Compounds of formula II may be prepared by coupling of compounds of formula XII with compounds of formula XIV at a temperature in the range of 0° C. to 150° C., preferably in the range of 20° C. to 80° C. in the presence of a solvent, for example TB:F, DCM, NMP, DCM/water (i.e. a two phase system) or DMF, optionally in the presence of a suitable inorganic or organic base, e.g. DIPEA or TEA, and a standard amide coupling reagent, e.g. HATU, TBTU, TFFH, PyBroP, EDC, or DCC, the latter two of which may optionally be polymer supported. Suitable additives such as HOBt and HOAt may optionally be utilised.

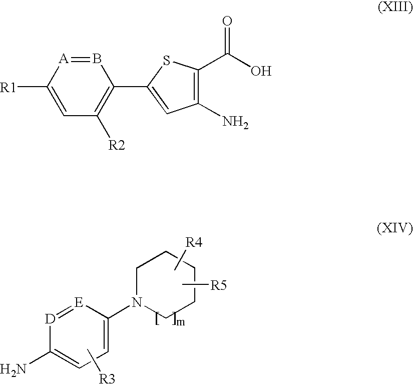

Persons skilled in the art will appreciate that in some cases, in order to obtain compounds of the invention, functional groups in compounds II-XIV (e.g. hydroxy groups or amino groups in R³, R⁴ or R⁵ or carboxylic acids groups) may require protection prior to the reactions described above. Amine protecting groups are known to those skilled in the art, for example the benzyl, t-Boc, or Cbz groups. Aromatic amino groups may also be masked as nitro groups during the reaction sequence. Hydroxy protecting groups are known to those skilled in the art, for example the t-butyl ether, TBDMS ether or THP, MEM or similar acetal type protecting groups. Carboxylic acid protecting groups are for example benzyl, t-butyl, ethyl or methyl esters.

Compounds of formulae II-XIV are either commercially available, known in the literature or can readily be prepared by methods known to those skilled in the art.

The compounds of the invention may be isolated from their reaction mixtures using conventional techniques. Stereoisomers may be separated using conventional techniques, e.g. chromatography or fractional crystallisation. Enantiomers may be isolated by separation of racemate for example by fractional crystallisation, resolution or HPLC. The diastereomers may be isolated by separation of isomer mixtures for instance by fractional crystallisation, HPLC or flash chromatography. Alternatively the stereoisomers may be made by chiral synthesis from chiral starting materials under conditions which will not cause racemisation or epimerisation, or by derivatisation, with a chiral reagent.

Persons skilled in the art will appreciate that, in order to obtain compounds of the invention in an alternative and in some occasions, more convenient manner, the individual process steps mentioned hereinbefore may be performed in a different order, and/or the individual reactions may be performed at a different stage in the overall route (i.e. chemical transformations may be performed upon different intermediates to those associated hereinbefore with a particular reaction).

Pharmaceutical Preparations

The compounds of the invention will normally be administered via the oral, parenteral, intravenous, intramuscular, subcutaneous or in other injectable ways, buccal, rectal, vaginal, transdermal and/or nasal route and/or via inhalation, in the form of pharmaceutical preparations comprising the active ingredient either as a free base, or a pharmaceutically acceptable inorganic or organic addition salt, in a pharmaceutically acceptable dosage form. Depending upon the disorder and patient to be treated and the route of administration, the compositions may be administered at varying doses.

Suitable daily doses of the compounds of the invention in the therapeutic treatment of humans are about 0.001-10 mg/kg body weight, preferably 0.01-3 mg/kg body weight.

Oral formulations are preferred particularly tablets or capsules which may be formulated by methods known to those skilled in the art to provide doses of the active compound in the range of 0.5 mg to 500 mg for example 1 mg, 3 mg, 5 mg, 10 mg, 25 mg, 50 mg, 100 mg and 250 mg.

According to a further aspect of the invention there is also provided a pharmaceutical formulation including any of the compounds of the invention, or pharmaceutically acceptable derivatives thereof, in admixture with pharmaceutically acceptable adjuvants, diluents and/or carriers.

The compounds of the invention may also be combined with other therapeutic agents, which are useful in the treatment of disorders associated with obesity, psychiatric disorders, neurological disorders and pain.

Pharmacological Properties

The compounds of formula I-Ia are useful for the treatment of obesity, psychiatric disorders such as psychotic disorders, anxiety, anxio-depressive disorders, depression, cognitive disorders, memory disorders, schizophrenia, epilepsy, and related conditions, and neurological disorders such as dementia, multiple sclerosis, Raynaud's syndrome, Parkinson's disease, Huntington's chorea and Alzheimer's disease. The compounds are also potentially useful for the treatment of immune, cardiovascular, reproductive and endocrine disorders, and diseases related to the respiratory and gastrointestinal systems. The compounds are also potentially useful as agents for ceasing consumption of tobacco, treating nicotine dependence and/or treating nicotine withdrawal symptoms, reducing the craving for nicotine and as anti-smoking agents. The compounds may also eliminate the increase in weight that normally accompanies the cessation of smoking. The compounds are also potentially useful as agents for treating or preventing diarrhea.

The compounds are also potentially useful as agents for reducing the craving/relapse for addictive substances that include, but are not limited to psychomotor-active agents such as nicotine, alcohol, cocaine, amphetamines, opiates, benzodiazepines and barbiturates. The compounds are also potentially useful as agents for treating drug addiction and/or drug abuse.

Accordingly, it is desirable to provide a compound and method of treatment which will be active in reducing craving for the abused substance, and which does not exacerbate the sympathetic response rate caused by the abused substance and which has favourable pharmacodynamic effects.

The compounds are also potentially useful as agents for treating pain disorders, including but not limited to acute and chronic nociceptive, inflammatory and neuropathic pain and migraine.

In another aspect the present invention provides a compound of formula I-Ia as claimed in any previous claim for use as a medicament.

In a further aspect the present invention provides the use of a compound of formula I-Ia in the preparation of a medicament for the treatment or prophylaxis of obesity, psychiatric disorders such as psychotic disorders, anxiety, anxio-depressive disorders, depression, bipolar disorder, ADHD, cognitive disorders, memory disorders, schizophrenia, epilepsy, and related conditions, neurological disorders such as dementia, multiple sclerosis, Parkinson's disease, Huntington's chorea and Alzheimer's disease and pain related disorders, including but not limited to acute and chronic nociceptive, inflammatory and neuropathic pain and migraine, comprising administering a pharmacologically effective amount of a compound of formula I-Ia to a patient in need thereof.

In a still further aspect the present invention provides a method of treating obesity, psychiatric disorders such as psychotic disorders, anxiety, anxio-depressive disorders, depression, bipolar disorder, ADHD, cognitive disorders, memory disorders, schizophrenia, epilepsy, and related conditions, and neurological disorders such as dementia, multiple sclerosis, Parkinson's disease, Huntington's chorea and Alzheimer's disease and pain related disorders, including but not limited to acute and chronic nociceptive, inflammatory and neuropathic pain and migraine, comprising administering a pharmacologically effective amount of a compound of Formula I-Ia to a patient in need thereof.

The compounds of the present invention are particularly suitable for the treatment of obesity.

In another aspect the present invention provides a method of treating obesity, type II diabetes, Metabolic syndrome and a method of preventing type II diabetes comprising administering a pharmacologically effective amount of a compound of formula I-Ia to a patient in need thereof.

Combination Therapy

The compounds of the invention may be combined with another therapeutic agent that is useful in the treatment of disorders associated with the development and progress of atherosclerosis such as hypertension, hyperlipidaemias, dyslipidaemias, diabetes and obesity. For example, a compound of the present invention may be used in combination with a compound that affects thermogenesis, lipolysis, fat absorption, satiety, or gut motility. The compounds of the invention may be combined with another therapeutic agent that decreases the ratio of LDL:HDL or an agent that causes a decrease in circulating levels of LDL-cholesterol. In patients with diabetes mellitus the compounds of the invention may also be combined with therapeutic agents used to treat complications related to micro-angiopathies.

The compounds of the invention may be used alongside other therapies for the treatment of metabolic syndrome or type 2 diabetes and its associated complications; these include biguanide drugs, insulin (synthetic insulin analogues), oral antihyperglycemics (these are divided into prandial glucose regulators and alpha-glucosidase inhibitors) and PPAR modulating agents.

In another aspect of the invention, the compound of formula I-Ia, or a pharmaceutically acceptable salt, solvate, solvate of such a salt or a prodrug thereof, may be administered in association with a PPAR modulating agent. PPAR modulating agents include but are not limited to a PPAR alpha and/or gamma agonist, or pharmaceutically acceptable salts, solvates, solvates of such salts or prodrugs thereof. Suitable PPAR alpha and/or gamma agonists, pharmaceutically acceptable salts, solvates, solvates of such salts or prodrugs thereof are well known in the art.

In addition the combination of the invention may be used in conjunction with a sulfonylurea. The present invention also includes a compound of the present invention in combination with a cholesterol lowering agent. The cholesterol lowering agents referred to in this application include but are not limited to inhibitors of HMG-CoA reductase (3-hydroxy-3-methylglutaryl coenzyme A reductase). Suitably the HMG-CoA reductase inhibitor is a statin.

In the present application, the term “cholesterol-lowering agent” also includes chemical modifications of the HMG-CoA reductase inhibitors, such as esters, prodrugs and metabolites, whether active or inactive.

The present invention also includes a compound of the present invention in combination with an inhibitor of the ileal bile acid transport system (IBAT inhibitor). The present invention also includes a compound of the present invention in combination with a bile acid binding resin.

According to an additional further aspect of the present invention there is provided a combination treatment comprising the administration of an effective amount of a compound of the formula I-Ia, or a pharmaceutically acceptable salt, solvate, solvate of such a salt or a prodrug thereof, optionally together with a pharmaceutically acceptable diluent or carrier, with the simultaneous, sequential or separate administration one or more of the following agents selected from:
a CETP (cholesteryl ester transfer protein) inhibitor;
a cholesterol absorption antagonist;
a MTP (microsomal transfer protein) inhibitor;
a nicotinic acid derivative, including slow release and combination products;
a phytosterol compound;
probucol;
an anti-obesity compound, for example orlistat (EP 129 748) and sibutramine (GB 2,184,122 and U.S. Pat. No. 4,929,629);
an antihypertensive compound, for example an angiotensin converting enzyme (ACE) inhibitor, an angiotensin II receptor antagonist, an andrenergic blocker, an alpha andrenergic blocker, a beta andrenergic blocker, a mixed alpha/beta andrenergic blocker, an andrenergic stimulant, calcium channel blocker, an AT-1 receptor blocker, a saluretic, a diuretic or a vasodilator;
a CB1 antagonist or inverse agonist, for example rimonabant;
another melanin concentrating hormone receptor I (MCHr1) antagonist;
a PDK inhibitor; or
modulators of nuclear receptors for example LXR, FXR, RXR, and RORalpha;
an SSRI;
a serotonin antagonist;
or a pharmaceutically acceptable salt, solvate, solvate of such a salt or a prodrug thereof, optionally together with a pharmaceutically acceptable diluent or carrier to a warm-blooded animal, such as man in need of such therapeutic treatment.

Therefore in an additional feature of the invention, there is provided a method for the treatment of type 2 diabetes and its associated complications in a warm-blooded animal, such as man, in need of such treatment which comprises administering to said animal an effective amount of a compound of formula I-Ia, or a pharmaceutically acceptable salt, solvate, solvate of such a salt or a prodrug thereof in simultaneous, sequential or separate administration with an effective amount of a compound from one of the other classes of compounds described in this combination section, or a pharmaceutically acceptable salt, solvate, solvate of such a salt or a prodrug thereof.

Therefore in an additional feature of the invention, there is provided a method of treating hyperlipidemic conditions in a warm-blooded animal, such as man, in need of such treatment which comprises administering to said animal an effective amount of a compound of formula I-Ia, or a pharmaceutically acceptable salt, solvate, solvate of such a salt or a prodrug thereof in simultaneous, sequential or separate administration with an effective amount of a compound from one of the other classes of compounds described in this combination section or a pharmaceutically acceptable salt, solvate, solvate of such a salt or a prodrug thereof.

According to a further aspect of the invention there is provided a pharmaceutical composition which comprises a compound of formula I-Ia, or a pharmaceutically acceptable salt, solvate, solvate of such a salt or a prodrug thereof, and a compound from one of the other classes of compounds described in this combination section or a pharmaceutically acceptable salt, solvate, solvate of such a salt or a prodrug thereof, in association with a pharmaceutically acceptable diluent or carrier.

According to a further aspect of the present invention there is provided a kit comprising a compound of formula I-Ia, or a pharmaceutically acceptable salt, solvate, solvate of such a salt or a prodrug thereof, and a compound from one of the other classes of compounds described in this combination section or a pharmaceutically acceptable salt, solvate, solvate of such a salt or a prodrug thereof.

According to a further aspect of the present invention there is provided a kit comprising:
a) a compound of formula I-Ia, or a pharmaceutically acceptable salt, solvate, solvate of such a salt or a prodrug thereof, in a first unit dosage form;
b) a compound from one of the other classes of compounds described in this combination section or a pharmaceutically acceptable salt, solvate, solvate of such a salt or a prodrug thereof; in a second unit dosage form; and
c) container means for containing said first and second dosage forms.

According to a further aspect of the present invention there is provided a kit comprising:
a) a compound of formula I-la, or a pharmaceutically acceptable salt, solvate, solvate of such a salt or a prodrug thereof, together with a pharmaceutically acceptable diluent or carrier, in a first unit dosage form;
b) a compound from one of the other classes of compounds described in this combination section or a pharmaceutically acceptable salt, solvate, solvate of such a salt or a prodrug thereof, in a second unit dosage form; and
c) container means for containing said first and second dosage forms.

According to another feature of the invention there is provided the use of a compound of the formula I-Ia, or a pharmaceutically acceptable salt, solvate, solvate of such a salt or a prodrug thereof, and one of the other compounds described in this combination section, or a pharmaceutically acceptable salt, solvate, solvate of such a salt or a prodrug thereof, in the manufacture of a medicament for use in the treatment of metabolic syndrome or type 2 diabetes and its associated complications in a warm-blooded animal, such as man.

According to another feature of the invention there is provided the use of a compound of the formula I-Ia, or a pharmaceutically acceptable salt, solvate, solvate of such a salt or a prodrug thereof, and one of the other compounds described in this combination section, or a pharmaceutically acceptable salt, solvate, solvate of such a salt or a prodrug thereof, in the manufacture of a medicament for use in the treatment of hyperlipidemic conditions in a warm-blooded animal, such as man.

According to a further aspect of the present invention there is provided a combination treatment comprising the administration of an effective amount of a compound of the formula I-Ia, or a pharmaceutically acceptable salt, solvate, solvate of such a salt or a prodrug thereof, optionally together with a pharmaceutically acceptable diluent or carrier, with the simultaneous, sequential or separate administration of an effective amount of one of the other compounds described in this combination section, or a pharmaceutically acceptable salt, solvate, solvate of such a salt or a prodrug thereof, optionally together with a pharmaceutically acceptable diluent or carrier to a warm-blooded animal, such as man in need of such therapeutic treatment.

Experimental Section

The invention will now be described in more detail with the following examples that are not to be construed as limiting the invention.

### Abbreviations:

Ac acetyl
BSA bovine serum albumin
Bu butyl
t-Boc tert-butyloxycarbonyl
CHO Chinese hamster ovary (cells)
DCM methylene chloride, CH₂Cl₂
DIPEA N,N-Diisopropylethylamine
DMF N,N-dimethylformamide
DMSO dimethylsulfoxide
DTT dithiothreitol
EDC 1-Ethyl-3-(3-dimethylaminopropyl)carbodiimide hydrochloride
EDTA ethylenediamine tetraacetic acid
ELS evaporative light scattering
ESI electrospray ionization
Et ethyl
GDP guanosine 5′-diphosphate
GPCR G-protein coupled receptor
GTP guanosine triphosphate
HATU O-(azabenzotriazol-1-yl)-N,N,N′,N′-tetramethyluronium hexafluorophosphate
hERG human ether-a-go-go related gene (potassium ion channel)
HEPES N-2-hydroxyethyl piperazine-N′-2-ethanesulfonic acid
HPLC high performance liquid chromatography
HOAt 1-Hydroxy-7-azabenzotriazole
LC liquid chromatography
MS mass spectroscopy
NMP N-methyl-pyrrolidinone
PyBroP Bromo-tris-pyrrolidino-phosphonium hexafluorophosphate
TBTU N,N,N′,N′-tetramethyl-O-(benzotriazol-1-yl)uronium tetrafluoroborate
TEA triethylamine
TFA trifluoroacetic acid
THF tetrahydrofuran
Tris trishydroxymethylaminomethane
t tert
rt. room temperature
sat. saturated
br broad
bs broad singlet
d doublet
dd doublet of doublets
dt doublet of triplets
m multiplet
q quartet
s singlet
t triplet

General Experimental Procedures

Flash column chromatography employed MERCK normal phase silica gel 60 Å (40-63 μm) or a Biotage Horizon Pioneer® HPFC system equipped with FLASH 12+M or FLASH 25+M or 40+M silica cartridges. Mass spectra were recorded on a Waters Micromass ZQ single quadrupole equipped with a pneumatically assisted electrospray interface (LC-MS).

HPLC analyses were performed on a Gynkotek P580 HPG, gradient pump with a Gynkotek UVD 170S UV-Vis detector. Column: Chromolith Performance RP-18e, 4.6×100 mm, Mobile phase A: Acetonitrile, Mobile phase B: 0.1% TFA (aq), Flow: 3 mL/min, Injection volume: 20 μl, Detection: 254 and 275 nm.

Purifications were performed on a semi preparative HPLC, Shimadzu LC-8A, Shimadzu SPD-10A UV-vis. detector equipped with a Waters X-terra® Prep MS C₁₈ Column, 250 mm×50 mm (10 μm) or on a Waters Prep LC 2000 with UV-detection, equipped with a Kromasil 10 μm C8 250 mm×20 nun column, or on a semi preparative HPLC, Shimadzu LC-8A, Shimadzu SPD-10A UV-vis.-detector equipped with a Waters Symmetry® 100 mm×19 mm C18 5 μm column.

¹H NMR and ¹³C NMR spectra were obtained at 298 K on a Varian Unity Plus 400 mHz, or a Varian Inova 500 MHz or a Varian Unity Plus 600 MHz or a Bruker Avance 300 MHz or Varian Gemini 2000 300 MHz. Chemical shifts are given in ppm with the solvent residual peak as internal standard: CDCl₃ δ_{H} 7.26, δ_{C} 77.2; MeOH-d₄ δ_{H} 3.31, δ_{H} 49.0; DMSO-d₆ δ_{H} 2.50; δ_{C} 39.5 ppm.

Microwave heating was performed using single node heating in a Smith Creator from Personal Chemistry, Uppsala, Sweden.

Chemical names (IUPAC) were generated using the software ACD/Name version 8.05. Names/reference numbers of starting materials (CAS no), either commercially available or prepared according to literature procedures.
(3R)-1-(4-nitro-2-methoxyphenyl)pyrrolidin-3-ol, 851690-75-0; methyl 3-amino-5-(4-chlorophenyl)thiophene-2-carboxylate, 91076-93-6; methyl 5-(4-chlorophenyl)-3-{[(1E)-(dimethylamino)methylene]amino}thiophene-2-carboxylate, 515141-52-3; (R)-(+)-3-pyrrolidinol, 2799-21-5; 5-bromo-2-nitropyridine, 39856-50-3; (S)-(−)-3-pyrrolidinol, 100243-39-8; 3-bromo-pyridine, 626-55-1; 5-(4-chlorophenyl)thiophene-2-carboxylic acid, 40133-14-0.

### WORKING EXAMPLES

### Example 1

### 6-(4-Chlorophenyl)-3-{4-[(3R)-3-hydroxypyrrolidin-1-yl]-3-methoxyphenyl}thieno[3,2-d][1,2,3]triazin4(3D)-one

### a) 3-Amino-5-(4-chlorophenyl)thiophene -2-carboxylic acid

Methyl 3-amino-5-(4-chlorophenyl)thiophene-2-carboxylate (2.00 g, 7.47 mmol) was refluxed in a solution of KOH (2.0 g, 36 mmol) in 50 mL of water and 50 mL of MeOH for 2 h. The MeOH was evaporated and the residue was diluted to the double volume with water and washed with ethyl acetate. The aqueous layer was acidified with NaHSO₄ (aq) and the precipitate was filtered, washed with water and dried to give 1.85 g (98%) of the desired compound.

¹H NMR (DMSO-d₆) δ 7.62 (m, 2H), 7.48 (m, 2H), 6.96 (s, 1H).

### b) (3R)-1-(4-amino-2-methoxyphenyl)pyrrolidin-3-ol

(3R)-1-(4-nitro-2-methoxyphenyl)pyrrolidin-3-ol (0.281 g, 1.18 mmol) was dissolved in 20 mL of dioxane and 50 mg of Pd(OH)₂/C was added. The nitro compound was hydrogenated at 3 atm for 4 h. The mixture was filtered and the catalyst washed with dioxane. The combined filtrate was evaporated and the product was used in step c) without further purification.

MS (ESI) 209 (M+1H⁺).

### c) 3-Amino-5-(4-chlorophenyl)N-{4-[(3R)-3-hydroxypyrrolidin-1-yl]-3-methoxyphenyl}thiophene -2-carboxamide

3-Amino-5-(4-chlorophenyl)thiophene-2-carboxylic acid (0.300 g, 1.18 mmol) was dissolved in 10 mL of NMP. HATU (0.562 g, 1.48 mmol) and DIPEA (0.62 mL, 3.5 mmol) were added. The reaction was stirred for 4 h and (3R)-1-(4-amino-2-methoxyphenyl)pyrrolidin-3-ol (0.246 g, 1.18 mmol) was added. The reaction mixture was heated to 80° C. for 4 h and was then poured into 100 mL of water and made alkaline with NaHCO₃ (aq). The mixture was extracted three times with EtOAc and the combined organic layer was washed with water, dried over Na₂SO₄ and evaporated. The residue was flash chromatographed on silica gel with DCM/MeOH 95/5. The material was recrystallised (partly dissolved) from MeOH to become pure. Yield: 0.300 g (57%).

¹H NMR (DMSO-d₆) δ 9.07 (s, 1H), 7.61 (d, 2H), 7.49 (d, 2H), 7.25 (m, 1H), 7.12 (m, 1H), 6.98 (s, 1H), 6.60-6.50 (m, 3H), 4.76 (bd, 1H), 4.26 (m, 1H), 3.69 (s, 3H), 3.45 (m, 1H), 3.10 (m, 1H), 2.95 (m, 1H), 1.93 (m, 1H), 1.71 (m, 1H).

### d) 6-(4-Chlorophenyl)-3-{4-[(3R)-3-hydroxypyrrolidin-1-yl]-3-methoxyphenyl}thieno[3,2-d][1,2,3]triazin-4(311)-one

3-Amino-5-(4-chlorophenyl)-N-{4-[(3R)-3-hydroxypyrrolidin-1-yl]-3-methoxyphenyl}thiophene-2-carboxamide (0.150 g, 0.338 mmol) was dissolved in 5 mL of acetic acid and 1 mL of water. Sodium nitrite (26 mg, 0.38 mmol) was added and the reaction was stirred for 45 min. The reaction mixture was poured into 50 mL of water and made alkaline with 1M NaOH whereafter it was extracted three times with DCM. The combined organic layer was washed with water, dried over Na₂SO₄ and evaporated. The residue was recrystallised from DMSO/MeOH. The product was further purified by prep HPLC (Chromasil C8 50×300 mm) using CH₃CN/0.1M NH₄OAc 30/70→100/0. The pertinent fractions were evaporated and freeze dried from DMSO to give 9.5 mg (6.2%) of the title compound.

¹H NMR (DMSO-d₆) δ 8.34 (s, 1H), 7.95 (d, 2H), 7.57 (d, 2H), 7.12 (bs, 1H), 7.03 (m, 1H), 6.69 (d, 1H), 4.85 (broad, 1H), 4.30 (broad, 1H), 3.72 (s, 3H), 3.60 (m, 1H), 3.45 (m, 1H), 3.15 (m, 1H), 1.94 (m, 1H), 1.79 (m, 1H).

MS (ESI) 455/457 (M+1H⁺).

### Example 2

### 6-(4-Chlorophenyl)3-{3-(hydroxymethyl)-4-[(3R)-3-hydroxypyrrolidin-1-yl]phenyl}thieno[3,2-d][1,2,3]triazin4(3H)-one

### a) (3R)-1-[2-(hydroxymethyl)4-nitrophenyl]pyrrolidin-3-ol

2-Chloro-5-nitrobenzylalcohol (4.0 g, 21 mmol) was added to (R)-3-pyrrolidinol and the neat mixture was stirred at 100° C. for 20 h. To the cool mixture was added TEA (2.9 mL, 21 mmol) and the mixture was purified by column chromatography on silica gel eluting with DCM/MeOH (10/2). The residue was washed with diethyl ether and water and the solids were filtered off to give 4.4 g (87%) of the desired product.

¹H-NMR (400 MHz, DMSO-d₆): δ 8.1 (d, 1H), 7.9 (dd, 1H), 6.63 (d, 1H), 5.35 (t, 1H), 5.0 (d, 1H), 4.6-4.5 (m, 2H), 4.32 (bs, 1H), 3.35-3.8 (m, 4H), 2.0-1.8 (m, 2H).

### b) (3R)-1-[4-amino-2-(hydroxymethyl)phenyl]pyrrolidin-3-ol

5 (3R)-1-[2-(hydroxymethyl)-4-nitrophenyl]pyrrolidin-3-ol (1.15 g, 4.82 mmol) was dissolved in 40 mL of dioxane and 130 mg of Pd(OM)₂/C was added. The nitro compound was hydrogenated at 3 atm for 4 h. The mixture was filtered and the catalyst washed with dioxane. The combined filtrate was evaporated and the product was used in step c without further purification.

MS (ESI) 209 (M+1H⁺).

### c) -Amino-5-(4-chlorophenyl)-N-{3-(hydroxymethyl)-4-1(3R)-3-hydroxypyrrolidin-1-yl]phenyl}thiophene -2-carboxamide

3-Amino-5-(4-chlorophenyl)thiophene-2-carboxylic acid (1.2 g, 4.8 mmol), HATU (2.28 g, 6.0 mmol) and DIPEA (1.86 g, 14.4 mmol) were added to 400 mL of NMP. The reaction was stirred for 50 min and (3R)-1-[4-amino-2-(hydroxymethyl)phenyl]pyrrolidin-3-ol (1.0 g, 4.8 mmol) was added. The reaction mixture was heated to 80° C. for 3 h and was then poured into 100 mL of water. The solids were filtered off and the yield was refluxed in acetonitrile for 30 min and the product was isolated by filtration to give 0.75 g (35%) of the title compound.

¹H-NMR (400 MHz, DMSO-d₆): δ 9.15 (s, 1H), 7.7-7.6 (m, 3H), 7.38 (d, 2H), 7.35 (d, 1H), 6.98 (s, 1H), 6.75 (d, 1H), 6.57 (s, 2H), 5.0 (bs, 1H), 4.8 (d, 1H), 4.44 (bs, 2H), 4.27 (bs, 1H), 3.3-3.15 (m, 1H), 3.1-2.85 (m, 2H), 2.1-1.7 (m, 2H).

### d) 6-(4-Chlorophenyl)-3-{3-(hydroxymethyl)-4-[(3R)-3-hydroxypyrrolidin-1-yl]phenyl}thieno[3,2-d] [1,2,3]triazin4(31H)-one

3-Amino-5-(4-chlorophenyl)-N-{3-(hydroxymethyl)-4-[(3R)-3-hydroxypyrrolidin-1-yl]phenyl}thiophene-2-carboxamide (0.6 g, 1.35 mmol) was dissolved in 12 mL of acetic acid and 2.4 mL of water and the mixture was cooled on icebath. Sodium nitrite (100 mg, 1.44 mmol) solved in water (1 mL) was added and the reaction was stirred for 15 min at 0° C. The reaction mixture was poured into 100 mL of ice-water and the solids were filtered off. The crude product was purified by column chromatography on silica gel eluting with DCM/acetone (gradient 10/3-10/8). The product was treated with MeOH and the solids were filtered off to give 0.3 g (49%) of the title compound.

¹H-NMR (400 MHz, DMSO-d₆): δ 8.47 (s, 1H), 8.06 (d, 2H), 7.69 (d, 2H), 7.63 (d, 1H), 7.42 (dd, 1H), 6.95 (d, 1H), 5.29 (t, 1H), 5.0 (d, 1H), 4.7-4.6 (m, 2H), 4.42 (bs, 1H), 3.65-3.5 (m, 2H), 3.3-3.15 (m, 2H), 2.15-1.85 (m, 2H)

### Example 3

### 6-(4-Chlorophenyl)3-{6-[(3R)-3-hydroxypyrrolidin-1-yl]pyridin-3-yl}thieno[3,2-d]pyrimidin-4(3R)-one

### a) (3R)-1-(5-nitropyridin-2-yl)pyrrolidin-3-ol

A mixture of 2-chloro-5-nitropyridine (2 g, 0.013 mol) and (R)-(+)-3-pyrrolidinol (2.2 g, 0.025 mol) was warmed to 100° C. for 12 hours. After completion of the reaction, it was allowed to cooled down to rt. The reaction was diluted with DCM (50 mL) and 1 N aq. NaOH (50 mL). The aqueous layer was extracted with DCM (2×50 mL). The combined organic phase was washed with brine, dried over anhydrous Na₂SO₄ and concentrated to get the title as yellow solid, 2.5 g (95%).

¹H-NMR (400 MHz, CDCl₃): δ 9.09 (d, 1H), 8.23 (dd, 1H), 6.37 (d,1H), 4.71 (s, 1H), 3.75 (bs, 5H), 2.18 (bs, 2H).

### b) (3R)-1-(5-aminopyridin-2-yl)pyrrolidin-3-ol

(3R)-1-(5-nitropyridin-2-yl)pyrrolidin-3-ol (0.3 g, 1.43 mmol) was dissolved in 10 mL of dioxane and 30 mg of Pd(OH)₂/C was added. The nitro compound was hydrogenated at 3 atm for 3 h. The mixture was filtered and the catalyst washed with dioxane. The combined filtrate was evaporated and the product was used in step without further purification.

MS (ESI) 180 (M+1H⁺).

### c) 6-(4-Chlorophenyl)-3-{6-[(3R)-3-hydroxypyrrolidin-1-yl]pyridin-3-yl}thieno[3,2-d]pyrimidin4(3H)-one

(3R)-1-(5-aminopyridin-2-yl)pyrrolidin-3-ol (1.43 mmol) and methyl 5-(4-chlorophenyl)-3-{[(1E)-(dimethylamino)methylene]amino)thiophene-2-carboxylate (0.46 g, 1.43 mmol) were added to phenol (1 g) and the reaction mixture was stirred at 120° C. for 1 h. To the cool mixture was added diethyl ether (10 mL). The precipitate was filtered off, washed with diethyl ether and acetone and dried to give 103 mg (17%) of the title compound.

¹H-NMR (400 MHz, DMSO-d₆): δ 8.35 (s, 1H), 8.12 (d, 1H), 7.94 (s, 1H), 7.88 (d, 2H), 7.61 (dd, 1H), 7.54 (d, 2H), 6.52 (d, 1H), 4.95 (d, 1H), 4.38 (bs, 1H), 3.55-3.3 (m, 4H), 2.1-1.85 (m, 2H).

### Example 4

### 6-(4-Chlorophenyl)-3-{5-[(3R)-3-hydroxypyrrolidin-1-yl]pyridin-2-yl}thieno[3,2-d]pyrimidin4(3B)-one

### a) (3R)-1-(6-nitropyridin-3-yl)pyrrolidin-3-ol

A mixture of 5-bromo-2-nitro pyridine (2 g, 0.0098 mol) and (R)-(+)-3-pyrrolidinol (1.7 g, 0.0197 mol) was warmed to 100° C. under inert atmosphere for 12 hours. After completion of the reaction, it was allowed to come to rt, diluted with DCM (50 mL) and 1 N NaOH (50 mL). The two layers were separated. The organic layer was washed with brine, dried over anhydrous Na₂SO₄ and concentrated. The crude product was purified by column chromatography using 70% EtOAc in petroleum ether as eluent. The product obtained after concentrating pure fractions was further washed with hexane to get the title compound as a brown solid, 1.4 g (68%).

¹H-NMR (400 MHz, MeOD): δ 8.25 (d, 1H), 7.86 (d, 1H), 7.15 (dd, 1H), 4.64 (bs, 1H), 3.75-3.5 (m, 4H), 2.3-2.1 (m,2H).

### b) (3R)-1-(6-aminopyridin-3-yl)pyrrolidin-3-ol

(3R)-1-(6-nitropyridin-3-yl)pyrrolidin-3-ol (0.4 g, 1.91 mmol) was dissolved in 10 mL of dioxane and 180 mg of Pd(OH)₂/C was added. The nitro compound was hydrogenated at 3 atm for 4 h. The mixture was filtered and the catalyst washed with dioxane. The combined filtrate was evaporated and the product was used in step without further purification.

MS (ESI) 180 (M+1H⁺).

### c) 6-(4-Chlorophenyl)-3-{5-[(3R)-3-hydroxypyrrolidin-1-yl]pyridin-2-yl}thieno[3,2-d]pyrimidin-4(3H) -one

(3R)-1-(6-aminopyridin-3-yl)pyrrolidin-3-ol (1.91 mmol) and methyl 5-(4-chlorophenyl)-3-{[(1E)-(dimethylamino)methylene]amino}thiophene-2-carboxylate (0.62 g, 1.91 mmol) were added to phenol (1.2 g) and the reaction mixture was stirred at 120° C. for 1 h 45 min. To the cool mixture was added diethyl ether (15 mL). The precipitate was filtered off and the yield was purified by column chromatography on silica gel eluting with DCM/MeOH (10%). The product was refluxed in aceton and the solids were filtered off to give 85 mg (10%) of the title compound.

¹H-NMR (400 MHz, DMSO-d₆): δ 8.46 (s, 1H), 7.93 (s, 1H), 7.89 (d, 1H), 7.84 (d, 1H), 7.55 (d, 2H), 7.48 (d, 1H), 7.07 (dd, 1H), 5.0 (d, 1H), 4.41 (bs, 1H), 3.5-3.35 (m, 3H), 3.2-3.1 (m, 1H), 2.15-1.85 (m, 2H).

### Example 5

### 6-(4-Chlorophenyl)3-{5-[(3R)-3-hydroxy-3-methylpyrrolidin-1-yl]pyridin-2-yl}thieno[3,2-d]pyrimidin-4(3H) -one

a) tert-Butyl (3S)-3-hydroxypyrrolidine-1-carboxylate

To a solution of (5)-3-pyrrolidinol (6 g, 0.068 mol) in NaOH solution (200 mL, 20%) at 0° C. was added Boc anhydride (15 g, 0.068 mol) dropwise over a period of 30 min. and stirred at 0° C. for 2.5 h. The reaction mixture was then slowly warmed to The reaction mixture was extracted with ethyl acetate (3×100 mL), washed with brine, dried over Na₂SO₄ and concentrated. The crude product was purified by column chromatography on silica gel using 50% EtOAc in pet. ether. Yield: 11.2 g (87%). The product was used directly in step b).

### b) tert-Butyl 3-oxopyrrolidine-1-carboxylate

To a solution of step a) product (10 g, 0.053 mol) in dry CH₂Cl₂ (200 mL) was added Dess-Martin periodinane (45.3 g, 0.106 mol) at 0° C. under nitrogen atm and stirred at RT for 2 days. To the reaction mixture was added sodium thiosulphate solution and filtered. The two layers were separated and the aqueous layer was extracted with CH₂Cl₂ (2×100 mL). The combined organic layers were washed with 10% NaHCO₃ solution and brine, dried over Na₂SO₄ and concentrated. The crude product was purified by column chromatography using 30% EtOAc in pet. ether. Yield=8.0 g (81%). The product was used directly in step c).

### c) tert-Butyl3-hydroxy-3-methylpyrrolidine-1-carboxylate

Methyl magnesium iodide [prepared from magnesium metal (1.73 g, 0.071 mol) and methyl iodide (4.7 mL, 0.074 mol) in dry ether (50 mL)] was slowly added to the solution of step b) product (6.6 g, 0.036 mol) in dry ether (150 mL) at 0° C. under nitrogen atm. The reaction mixture was slowly warmed to RT and stirred for 1.5 h and after cooling to 0° C., it was quenched with saturated NH₄Cl solution. The two layers were separated and the aqueous layer was extracted with ethyl acetate (3×100 mL). The combined organic layers were washed with brine, dried over Na₂SO₄ and concentrated. The crude product was purified by column chromatography using 40% EtOAc in pet. ether. Yield=3.4 g (48%). The product was used directly in step d).

### d) 3-Methylpyrrolidin-3-ol hydrochloride

The step c) product (2.4 g, 0.0119 mol) was taken in diethyl ether saturated with HCl (50 mL) and stirred at RT for 5 h. The reaction mixture was concentrated. This HCl salt (1.6 g) was as such taken for the next step without purification in step e).

### e) 3-Methyl-1-(6-nitropyridin-3-yl)pyrrolidin-3-ol

A mixture of 5-bromo-2-nitropyridine (2 g, 0.010 mol), HCl salt of pyrrolidinol derivative (1.62 g, 0.012 mol) and dry K₂CO₃ (4 g, 0.030 mol) in dry DMF (25 mL) was heated at 120° C. for 12 h under nitrogen atm. The reaction mixture was brought to RT and filtered. The filtrate was concentrated, added with ethyl acetate and washed with water (3×100 mL) and brine, dried over Na₂SO₄ and concentrated. The crude product was purified by column chromatography using 50% EtOAc in pet. ether.Yield=1.1 g (50%).

¹H-NMR (400 MHz, CDCl₃): δ 8.16 (d, 1H), 7.79 (m, 1H), 6.83 (m, 1H), 3.70 (m, 1H), 3.57 (m, 1H), 3.46 (d, 1H), 3.42 (d, 1H), 2.05-2.20 (m, 2H), 1.57 (s, 3H).

### f) 3-Methyl-1-(6-amino pyridin-3-yl)pyrrolidin-3-ol

1-(6-nitropyridin-3-yl)-3-methyl-pyrrolidin-3-ol (0.200 g, 0.896 mmol) was dissolved in 10 mL of ethanol and 40 mg of 10% Pd/C was added. The nitro compound was hydrogenated at atmospheric pressure for 3.5 h. The mixture was filtered and the catalyst washed with ethanol. The combined filtrate was evaporated and the product was used in step without further purification.

MS (ESI) 194 (M+1H⁺).

### g) 6-(4-Chlorophenyl)-3-[5-(3-hydroxy-3-methylpyrrolidin-1-yl)pyridin-2-yl]thieno [3,2-d]pyrimidin-4(3I])-one

1-(6-aminopyridin-3-yl)-3-methyl-pyrrolidin-3-ol (0.160 g, 0.828 mmol) and methyl 5-(4-chlorophenyl)-3-{[(1E)-(dimethylamino)methylene]amino}thiophene-2-carboxylate (0.267 g, 0.828 mmol) were dissolved in 3 mL of methanol and the reaction mixture was heated in a microwave reactor at 140° C. for 10 min. The solvent was evaporated and the crude product was purified by prep HPLC (Chromasil C8 50×300 mm) using CH₃CN/0.2% HOAc 5/95→50/50. After freeze drying, 23 mg (6%) of the title compound as its free base was obtained.

1H-NMR (400 MHz, DMSO-d₆): δ 8.45 (s, 1H), 7.75-8.00 (m, 4H), 7.40-7.60 (m, 3H), 7.02 (m, 1H), 4.83 (s, 1H), 3.20-3.50 (m, 4H partially obscured by water in DMSO), 1.80-2.00 (m, 2H), 1.33 (s, 3H).

¹³C-NMR (100 MHz, DMSO-d₆): δ 158.0, 156.6, 150.7, 149.3, 144.5, 137.6, 135.0, 132.2, 132.0, 130.0, 128.6, 122.8, 122.7, 122.5, 119.7, 76.1, 61.1, 47.2, 41.1,26.4.

MS (ESI) 439/441 (M+1H⁺).

### Example 6

### 2-(4-Chlorophenyl)6-(5-[(3R)-3-hydroxypyrrolidin-1-yl]pyridin-2-yl]thieno[2,3-d]pyridazin-7(6H)-one

### a) (3R)-1-pyridin-3-ylpyrrolidin-3-ol

A mixture of (R)-3-pyrrolidinol (0.560 g, 6.43 mmol) and 3-bromopyridine (4.16 g, 26.3 mmol) was subjected to microwave heating at 220° C. for 5 h. 1M NaOH was added and the mixture extracted with DCM and EtOAc. The combined organic layers were dried with MgSO₄, filtered and concentrated. The residue was dissolved in toluene and concentrated several times to remove the excess of 3-bromopyridine. To the residue was added heptane and the mixture heated, cooled to rt and decanted, to give 0.36 g (34%/) of the title compound.

¹H NMR (MeOH-d₄) δ 7.79 (d, 1H, J=2.6 Hz), 7.74 (d, 1H, J=4.2 Hz), 7.15 (dd, 1H, J=8.4, 4.7 Hz), 6.90 (bd, 1H, J=8.4 Hz), 4.95 (bs, 1H), 3.46-3.35 (m, 2H), 3.28 (ddd, 1H, J=8.7, 8.7, 3.2 Hz), 3.17 (d, 1H, J=8.5 Hz), 2.15-1.96 (m, 2H).

¹³C NMR (MeOH-d₄) δ 145.6, 136.7, 133.8, 125.3, 119.8, 71.5, 56.5, 46.3, 34.7.

### b) (3R)-1-(6-bromopyridin-3-yl)pyrrolidin-3-ol

To a stirred solution of (3R)-1-pyridin-3-ylpyrrolidin-3-ol (0.62 g, 3.78 mmol) in DCM (50 mL) at 0° C. was added 2,4,4,6-tetrabromocyclohexa-2,5-dier-1-one (1.58 g, 3.85 mmol) dissolved in DCM (5 mL). The mixture was stirred at rt. over night. A second portion of 2,4,4,6-tetrabromocyclohexa-2,5-dien-1-one ( 0.40 g, 0.98 mmol) was added and the resulting mixture sirred for an additional 30 min. 1M NaOH was added and the phases separated. The organic layer was concentrated and the residue purified on C8-HPLC (0.1M NH₄OAc, gradient 5→100% CH₃CN) to give 0.316 g (34%) of the title compound.

¹H NMR (CDCl₃) δ 7.42 (d, 1H, J=3.0 Hz), 7.08 (d, 1H, J=8.7 Hz), 6.55 (dd, 1H, J=8.7, 3.0 Hz), 4.54 (bs, 1H), 4.33 (b, 1H), 3.37-3.28 (m, 2H), 3.19-3.10 (m, 2H), 2.07-1.99 (m, 2H).

¹³C NMR (CDCl₃) δ 143.1, 133.1, 127.6, 125.8, 121.3, 70.5, 55.9, 45.5, 34.0.

### c) 5-(4-chlorophenyl)3-formylthiophene-2-carboxylic acid

To a stirred solution 5-(4-chlorophenyl)thiophene-2-carboxylic acid (11 g, 0.046 mol) in dry THF (150 mL) was added n-BuLi (63 mL, 0.102 mol, 1.6 M solution in hexane) in a dropwise manner at −78° C. under inert atmosphere. The temperature was slowly raised to 0° C. over a period of 4 h. The reaction mixture was again cooled to −70° C. and dry DMF (34 mL, 0.43 mol) was added slowly. After completion of the addition of DMF, temperature was raised to −10° C. and stirred for 2 h. The reaction mixture was again cooled to −30° C. and 1.5 N HCl (50 mL) was added slowly and reaction was allowed to come to RT. The reaction mixture was extracted with EtOAc (4×200 mL). The combined organic layer was washed with water (2×150 mL), brine (2×150 mL), dried (Na2SO4) and concentrated. The crude product was washed with nether (100 mL) to afford 8 g of the title compound (65%). This was found pure enough (TLC, R_{f}=0.2 (CHCl₃:MeOH, 8:2)) to carry further. Since this intermediate is unstable it has to be used immediately in step d).

### d) 2-(4-Chlorophenyl)thieno[2,3-d]pyridazin-7(6R)-one

To a stirred solution of 5-(4-chlorophenyl)-3-formylthiophene-2-carboxylic acid (3 g, 0.011 mol) in ethanol (30 mL) was added dropwise hydrazine hydrate (0.65 mL, 0.013 mol). To this was added conc. HCl (1.8 mL, 0.058 mol) in a dropwise manner and heated to 82° C. for 2 days. The reaction mixture was allowed to cool down and 10% NaHCO₃ (5 mL) was added slowly until pH=8. The solid was filtered, washed with water (200 mL) and dried to afford 2.1 g of the title compound (71%).

¹H NMR (DMSO-d₆) δ 12.98 (bs, 1H), 8.39 (s, 1H), 7.98 (s, 1H), 7.88 (d, 2H, J=8.6 Hz), 7.59 (d, 2H, J=8.6 Hz).

### e) 2-(4-Chlorophenyl)-6-{5-[(3R)-3-hydroxypyrrolidin-1-yl]pyridin-2-yl}thieno[2,3-d]pyridazin-7(6H)-one

A mixture of 2-(4-chlorophenyl)thieno[2,3-d]pyridazin-7(6H)-one (0.192 g, 0.73 mmol), (3R)-1-(6-bromopyridin-3-yl)pyrrolidin-3-ol (0.192 g, 0.73 mmol, from step b), Cu₂O , trans-1,2-bis(methylamino)cyclohexane and K₃PO₄ in toluene (1 mL) under an atm of N₂, was stirred at 110° C. over night, followed by microwave heating for an additional 12 h at 150° C. The mixture was diluted with DCM/MeOH 5:1, filtered through Celite and concentrated. Purification on C8-HPLC (0.1% HOAc, gradient 30→100% CH₃CN) gave 0.021 g (33%) of the title compound.

¹H NMR (DMSO-d₆) δ 8.51 (s, 1H), 8.03 (s, 1H), 7.91 (d, 2H, J=8.7 Hz), 7.84 (d, 1H, J=2.6 Hz), 7.60 (d, 2H, J=8.7 Hz), 7.36 (d, 1H, J=8.7 Hz), 7.05 (dd, 1H, J=8.7, 2.8 Hz), 5.04 (br, 1H), 4.45 (bs, 1H), 3.49 (dd, 1H, J=10.4, 4.7 Hz), 3.46-3.30 (m, 2H, obscured by H₂O signal), 3.18 (bd, 1H, J=10.1 Hz), 2.13-1.90 (m, 2H).

¹³C NMR DMSO-d₆) δ 156.0, 150.8, 143.6, 141.4, 140.2, 135.5, 134.5, 133.3, 131.6, 130.9, 129.5, 128.3, 121.7, 120.8, 119.1, 69.2, 55.8, 45.4, 33.7.

MS (ESI+) 424.9(M+1H⁺).

Pharmacological Properties

MCHr1 Receptor Radioligand Binding.

Assays were performed on membranes prepared from CHO-K1 cells expressing the human Melanin concentrating hormone receptor 1 (hMCHr1, 5.45 pmol/mg protein; Euroscreen). Assays were performed in a 96-well plate format in a final reaction volume of 200 μl per well. Each well contained 6 μg of membrane proteins diluted in binding buffer (50 mM Tris, 3 mM MgCl₂, 0.05% bovine serum albumin and the radioligand ¹²⁵I-MCH (IM344 Amersham) was added to give 10 000 cpm (counts per minute) per well. Each well contained 2 μl of the appropriate concentration of competitive antagonist prepared in DMSO or in HOAc and left to stand at 30° C. for 60 minutes. Non-specific binding was determined as that remaining following incubation with 1 μM MCH (Melanin concentrating hormone, H-1482 Bachem). The reaction was terminated by transfer of the reaction to GF/A filters using a Micro96 Harvester (Skatron Instruments, Norway). Filters were washed with assay buffer. Radioligand retained on the filters was quantified using a 1450 Microbeta TRILUX (Wallac, Finland).

Non-specific binding was subtracted from all values determined. Maximum binding was that determined in the absence of any competitor following subtraction of the value determined for nonspecific binding. Binding of compounds at various concentrations was plotted according to the equation

y=A+((B−A)/1+((C/x)̂D)))

and IC₅₀ estimated where
A is the bottom plateau of the curve i.e. the final minimum y value
B is the top of the plateau of the curve i.e. the final maximum y value
C is the x value at the middle of the curve. This represents the log EC₅₀ value when A+B=100
D is the slope factor. x is the original known x values. y is the original known y values.

The compounds exemplified herein had an IC₅₀ of less than 100 nM in the abovementioned human MCHr binding assay. Preferred compounds had an activity of less than 20 nM. For instance, an IC₅₀ value of 11 nM was obtained for the compound of Example 1.

MCH1 Functional Assay

Membranes expressing recombinant hMCHr (5.45 pmol/mg protein; Euroscreen) were prepared in assay buffer (50 mM HEPES, 100 mM NaCl, 5 mM MgCl₂, 1 mM EDTA, 200 μM DTT, 20 μM GDP (Sigma) containing 0.1 μg/mL BSA, pH7.4) before assay. The assays were performed using membranes at 6 μg/well in an assay volume of 200 μL and the appropriate concentrations of compounds prepared in DMSO or in HOAc. The reaction was started by addition of 0.056 nM [³⁵S]GTPγS (Specific activity >1000 Ci/mmol;

Amersham) and an ED₈₀ concentration of MCH (determined for each membrane and each MCH batch). Non-specific binding was determined using 20 μM non-radiolabelled GTPγS. Plates were incubated for 45 min at 30° C. Free and bound GTPγS were separated by filtration binding using GF/B filter mats presoaked in wash buffer (50 mM Tris, 5 mM MgCl₂, 50 mM NaCl, pH 7.4) using a Micro96 cell harvester (Skatron Instruments) and the filters then dried at 50° C. before counting using a 1450 Microbeta TRILUX (Wallac).

Data are means±SD for experiments performed in triplicate. IC₅₀ values of antagonists were determined using nonlinear regression analysis of concentration response curves using Activity Base.

hERG Activity

hERG testing was performed using a modified version of the method described by Kiss L, Bennett P B, Uebele V N, Koblan K S, Kane S A, Neagle B, Schroeder K. “High throughput ion-channel pharmacology: planar-array-based voltage clamp.” Assay Drug Dev Technol. 1, 127-35. (2003). The compound of Example 1 had an IC₅₀ exceeding 5 μM in the abovementioned assay.

Diet Induced Obesity Model in Mouse

The utility of the compounds of the present invention in the treatment of obesity and related conditions is demonstrated by a decrease in body weight in cafeteria diet-induced obese mice. Female C57B1/6J mice were given ad libitum access to calorie-dense ‘cafeteria’ diet (soft chocolate/cocoa-type pastry, chocolate, fatty cheese and nougat) and standard lab chow for 8-10 weeks until a body weight of 45-50 grams was achieved. Compounds to be tested were then administered systemically (iv, ip, sc or po) once daily for a minimum of 5 days, and the body weights of the mice monitored on a daily basis.

During this period ad libitum access to calorie-dense ‘cafeteria’ diet and standard lab chow was maintained. Simultaneous assessment of adiposity was carried by means of DEXA imaging at baseline and termination of the study. Blood sampling was also carried out to assay changes in obesity-related plasma markers. Compounds of the invention induce significant decrease in body weight, with the major effect being via a reduction in fat-mass.

Compounds of the invention have the advantage that they may be more potent, more selective (e.g. vs. ion channels such as hERG and/or vs. GPCR's related to MCHr1) more efficacious in vivo, be less toxic, produce fewer side effects, be more easily absorbed, be less metabolised and/or have a better pharmacokinetic profile than, or have other useful pharmacological or physicochemical properties (e.g. solubility) over, compounds known in the prior art.

1. A compound of formula I 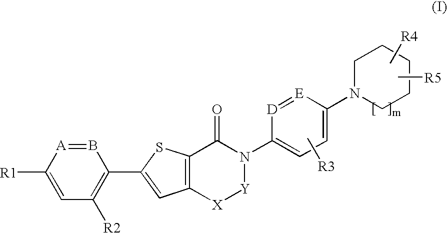
wherein
A and B independently represent C or N,
D and E independently represent C or N,
X—Y represents N═C (provided that at least one of A, B, D or E represents N), or
X—Y represents C═N, or
X represents NH and Y represents C═O, or
X—Y represents N═N,
R¹ and R² independently represent H, C₁₋₃ alkyl (optionally substituted with one or more F), C₁₋₃ alkoxy (optionally substituted with one or more F), Cl or F,
R³ represents H, F, Cl, cyano, hydroxy, C₁₋₃ alkoxy (optionally substituted with hydroxy, methoxy or with one or more F) or C₁₋₃ alkyl (optionally substituted with hydroxy, methoxy, amino, methylamino, dimethylamino or with one or more F),
R⁴ and R⁵ independently represent H, oxo, hydroxy, C₁₋₃ alkoxy (optionally substituted with hydroxy, methoxy or with one or more F), C₁₋₃ alkyl (optionally substituted with hydroxy, methoxy, amino, methylamino, dimethylamino or with one or more F) or C₁₋₃ acyloxy wherein the alkyl portion may optionally be substituted by one or more of methyl, amino, methylamino, dimethylamino or carboxy,
m is 0 or 1,
or a pharmaceutically acceptable salt thereof.

2. A compound according to claim 1, in which A, B and E all represent C, and D represents N.

3. A compound according to claim 1, in which X—Y represents C═N, or X—Y represents N═N.

4. A compound according to claim 1, in which A and B both represent C, D and E both represent C, X—Y represents C═N, or X—Y represents N═N.

5. A compound according to claim 1, in which X—Y represents N═C (provided that at least one of A, B, D or E represents N).

6. A compound according to claim 1, in which
A, B, D and E all represent C,
X—Y represents N═N,
R¹ represents Cl, F, CF₃, CHF₂, CH₂F, methyl, OCF₃ or OCHF₂,
R²represents H, Cl, F or CH₃,
R³ represents H, F, Cl, hydroxy, methoxy or hydroxymethyl, where the R³ substituent is placed in the meta position relative to the fused heterocyclic ring system,
R⁴ represents oxo, hydroxy, methoxy or hydroxymethyl,
m is 0, and wherein the R⁴ substituent is placed in position 3 of the pyrrolidine ring and
R⁵ represents H.

7. A compound according to claim 1, in which
A, B, and E represent C, and D represents N
X—Y represents N═C or C═N
R¹ represents Cl, F, CF₃, CHF₂, CH₂F, methyl, OCF₃ or OCHF₂,
R² represents H
R³ represents H
R⁴ represents hydroxy or hydroxymethyl,
m is 0, and wherein the R⁴ substituent is placed in position 3 of the pyrrolidine ring and
R⁵ represents H or methyl placed in the same position as R⁴,

8. One or more of the following compounds:
6-(4-chlorophenyl)-3-{4-[(3R)-3-hydroxypyrrolidin-1-yl]-3-methoxyphenyl}thieno[3,2-d][1,2,3]triazin-4(3H)-one,
6-(4-chlorophenyl)-3-{3-(hydroxymethyl)-4-[(3R)-3-hydroxypyrrolidin-1-yl]phenyl}thieno[3,2-d][1,2,3]triazin-4(3H)-one;
6-(4-chlorophenyl)-3-{6-[(3R)-3-hydroxypyrrolidin-1-yl]pyridin-3-yl}thieno[3,2-d]pyrimidin-4(3H)-one;
6-(4-chlorophenyl)-3-{5-[(3R)-3-hydroxypyrrolidin-1-yl]pyridin-2-yl}thieno[3,2-d]pyrimidin-4(3H)-one;
6-(4-chlorophenyl)-3-{5-[3-hydroxy-3-methylpyrrolidin-1-yl]pyridin-2-yl}thieno[3,2-d]pyrimidin-4(3H)-one;
2-(4-chlorophenyl)-6-{5-[(3R)-3-hydroxypyrrolidin-1-yl]pyridin-2-yl}thieno[2,3-d]pyridazin-7(6H)-one, and
or a pharmaceutically acceptable salt thereof.

9. (canceled)

10. A pharmaceutical formulation comprising a compound of formula I, as defined in claim 1 and a pharmaceutically acceptable adjuvant, diluent or carrier.

11-12. (canceled)

13. A method of treating obesity, a psychiatric disorder, anxiety, an anxio-depressive disorder, depression, bipolar disorder, ADHD, a cognitive disorder, a memory disorder, schizophrenia, epilepsy, a neurological disorder, or pain related disorder, comprising administering a pharmacologically effective amount of a compound as claimed in claim 1 to a patient in need thereof.

14. A method of treating obesity, type II diabetes, or metabolic syndrome comprising administering a pharmacologically effective amount of a compound as claimed in claim 1 to a patient in need thereof.
